(19) United States
(12) Patent Application Publication
(10) Pub. No.: US 2025/0135095 A1
(43) Pub. Date: May 1, 2025

(54) FLUIDIC SYSTEMS FOR OPHTHALMIC PROCEDURES

(71) Applicant: Alcon Inc., Fribourg (CH)

(72) Inventors: Vincent A. BAXTER, Temecula, CA (US); Justin PRATT, San Clemente, CA (US)

(21) Appl. No.: 18/929,909

(22) Filed: Oct. 29, 2024

Related U.S. Application Data
(60) Provisional application No. 63/594,924, filed on Oct. 31, 2023.

(51) Int. Cl. A61M 3/02; A61F 9/007

(52) U.S. Cl. CPC A61M 3/0258; A61F 9/007; A61B 2217/007; A61M 2205/502; A61M 2210/0612

(57) ABSTRACT

Embodiments disclosed herein provide a compression system including a squeeze plate, a linkage assembly, and a drive assembly. The squeeze plate is configured to engage with a fluid bag. The linkage assembly comprises a plurality of brackets each coupled to a plurality of linkage sets, where actuation of the linkage assembly causes lateral translation of the squeeze plate. The drive assembly comprises a lead screw disposed through each of the plurality of brackets, an actuator configured to rotate the lead screw to actuate the drive assembly, and a nut assembly coupled to the lead screw and at least one of the plurality of brackets, where the nut assembly is configured to provide a biasing force to maintain a target range of pressure on the fluid bag if the actuator fails.

### CROSS-REFERENCE TO RELATED APPLICATIONS

This application claims the benefit of U.S. Provisional Application Ser. No. 63/594,924 (filed on Oct. 31, 2023), the content of which is incorporated herein by reference in its entirety.

### INTRODUCTION

During certain surgical procedures, such as ophthalmic surgical procedures, a surgical console may be utilized to control the flow of fluids to and from a patient. To deliver fluids to the patient, many fluidic systems utilize a compression system disposed within, or in operable communication with, the surgical console to engage with a fluid-filled bag, or other reservoir, that is fluidically coupled with a fluid delivery device.

However, fluidic systems with electrically powered motor and compression mechanisms may experience power failures, or other similar events, which can disrupt the flow of fluids during a surgical procedure. For example, such systems may not be able to drive the compression mechanism engaged with the fluid-filled bag or other reservoir in the event of a loss of power, motor fault, control fault, or other similar event. As a result, a drastic change in pressure and/or fluid flow may occur since compression forces on the fluid-filled bag or other reservoir may not be maintained. Such events can lead to various patient-related complications, thereby reducing the safety and/or efficacy of the surgical procedure.

Further, some current fluidic systems employ a costly linear slide compression mechanism having a screw and motor assembly. The linear slide is typically oriented perpendicular to the fluid bag or reservoir to facilitate efficient compression and control. Because of its orientation, the linear slide requires that the compression system have a substantial lateral (e.g., horizontal) footprint in order to accommodate its screw and motor assembly, which increases the size of the compression system and, in many cases, the surgical console.

SUMMARY

The present disclosure relates generally to fluidic systems for surgical consoles.

In certain embodiments, a compression system is provided which is configured to be coupled to a surgical console for ophthalmic irrigation and/or infusion during a surgical procedure.

The compression system includes a squeeze plate configured to engage with a fluid bag, a linkage assembly comprising a plurality of brackets each coupled to a plurality of linkage sets, where actuation of the linkage assembly causes lateral translation of the squeeze plate, and a drive assembly comprising a lead screw disposed through each of the plurality of brackets, an actuator configured to rotate the lead screw to actuate the drive assembly, and a nut assembly coupled to the lead screw and at least one of the plurality of brackets, where the nut assembly is configured to provide a biasing force to maintain a target range of pressure on the fluid bag if the actuator fails.

In certain embodiments, another compression system is provided which is configured to be coupled to a surgical console for ophthalmic irrigation and/or infusion during a surgical procedure. The compression system includes a squeeze plate configured to engage with a fluid bag, a linkage assembly comprising a plurality of brackets each coupled to a plurality of linkage sets, where actuation of the linkage assembly causes lateral translation of the squeeze plate, and a drive assembly comprising a lead screw disposed through each of the plurality of brackets, an actuator configured to rotate the lead screw to actuate the drive assembly, and a nut assembly coupled to the lead screw and at least one of the plurality of brackets, where the plurality of brackets are configured to provide a biasing force to maintain target range of pressure on the fluid bag if the actuator fails.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of an ophthalmic surgical system that may be used to perform ophthalmic procedures on an eye, according to certain embodiments.

FIG. 1B is an example of subsystems of a console of the ophthalmic surgical system of FIG. 1A, according to certain embodiments.

FIG. 2 is a back side isometric view of an example fluidics module of a console of the ophthalmic surgical system of FIG. 1A, according to certain embodiments.

FIG. 3A is a back side isometric view of an example compression system of the fluidics module of FIG. 2, according to certain embodiments.

FIGS. 3B-3E are schematic side views of the compression system of FIG. 3A, according to certain embodiments.

FIG. 3F is a cross sectional view illustrating a lead screw and a nut assembly of the compression system of FIG. 3A, according to certain embodiments.

FIGS. 4A-4B are side views of the compression system of FIG. 3A illustrating lateral translation of a squeeze plate, according to certain embodiments.

FIG. 5 is a cross sectional view of another example compression system of the fluidics module of FIG. 2, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

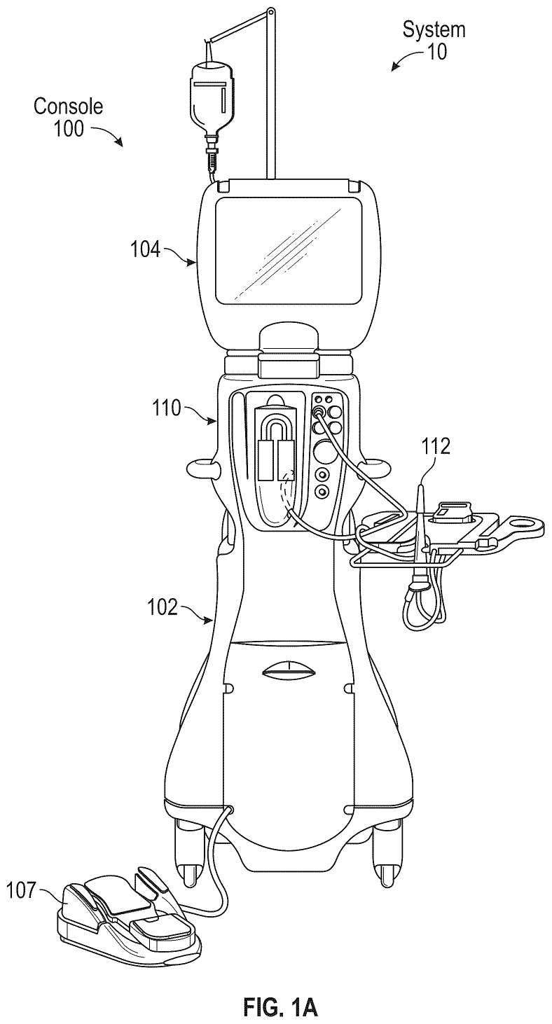

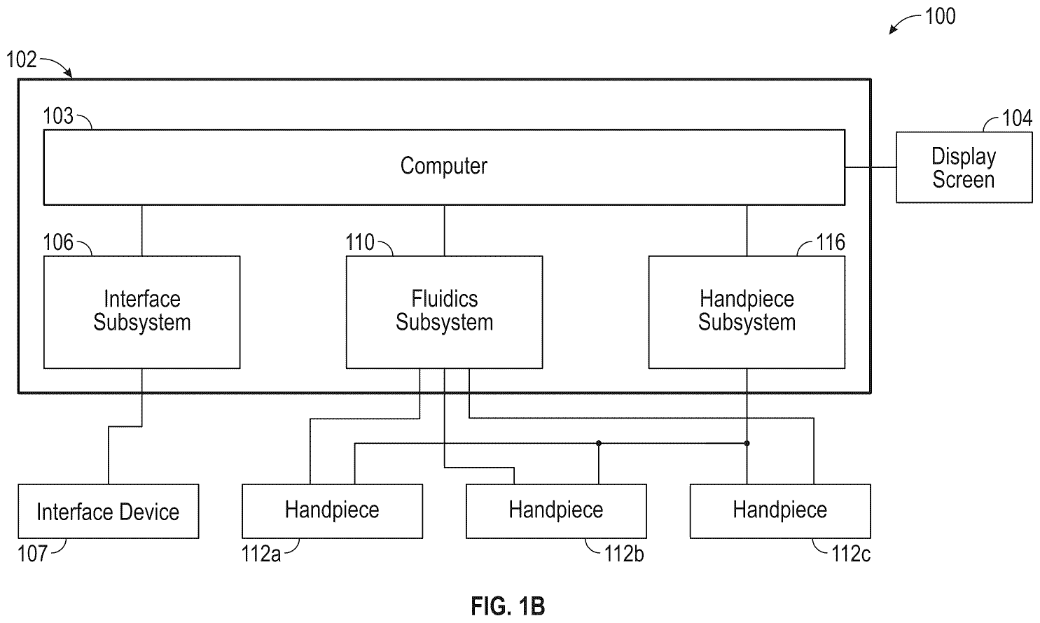

FIG. 1A illustrates an example of an ophthalmic surgical system 10 that may be used to perform ophthalmic procedures on an eye, according to certain embodiments. In the illustrated embodiments, system 10 includes console 100 (also referred to as a “surgical console”), an interface device 107 (e.g., a foot pedal), and a handpiece 112 (or other fluid delivery device). Console 100 includes a housing 102, a display screen 104, and a fluidics subsystem 110. The components of system 10 and console 100 may be mechanically, fluidically, and/or electrically coupled as shown and are described in more detail with reference to FIG. 1B.

FIG. 1B illustrates example subsystems of console 100 of ophthalmic surgical system 10 of FIG. 1A, according to certain embodiments. Console 100 includes housing 102, which accommodates a computer 103 (with an associated display screen 104) and subsystems 106, 110, and 116, which support interface device 107 and handpieces 112 (112a-c). An interface device 107 receives input to console 100 for controlling the operations of the console 100, provides output generated by the console 100, and/or processes the input and/or output. Although in FIG. 1A the interface device 107 is a foot pedal, other types of interface devices may include a manual input device (e.g., a keyboard), a display, etc. Interface subsystem 106 receives input from and/or provides output to interface device 107.

Computer 103 controls operation of ophthalmic surgical system 10. Generally, computer 103 includes a processor and a memory. The memory may include any device operable to receive, store, or recall data, including, but not limited to, electronic, magnetic, or optical memory, whether volatile or non-volatile. The memory may include code stored thereon. The code may include instructions that may be executable by the processor. The code may be created, for example, using any programming language, including but not limited to, C, C++, Java, Python, Rust, or any other programming language (including assembly languages, hardware description languages, and database programming languages). In some instances, the code may be a program that, when loaded into the processor, causes the surgical console 100 to receive and process information from one or more of subsystems 106, 110, or 116 for, e.g., providing fluid control for one or more handpieces 112 or other devices in communication with the surgical console 100.

The processor may be, or include, a microprocessor, a microcontroller, an embedded microcontroller, a programmable digital signal processor, or any other programmable device operable to receive information from the memory or other devices in communication with the processor, computer 103, and/or console 100, and perform one or more operations on the received information. For example, the processor may send instructions to components of fluidics subsystem 110, or other devices or systems in communication with computer 103, for controlling such devices and systems. The processor may also be operable to output results based on the operations performed thereby. A display screen 104 shows data provided by the processor of computer 103. In some instances, the processor may also be or include an application specific integrated circuit, a programmable gate array, programmable array logic, or any other device of combinations of devices operable to process electric signals.

Handpiece 112 may be any suitable ophthalmic surgical instrument, e.g., an ultrasonically-driven phacoemulsification (phaco) handpiece, a laser handpiece, an irrigating cannula, an infusion cannula, a vitrectomy handpiece, or another suitable surgical handpiece or fluid delivery device.

Handpiece subsystem 116 supports one or more handpieces 112. For example, handpiece subsystem 116 may manage ultrasonic oscillation for a phaco handpiece, provide laser energy to a laser handpiece, control operation of an irrigating cannula, infusion cannula, and/or manage features of a vitrectomy handpiece.

Fluidics subsystem 110 provides fluid control for one or more handpieces 112 (112a-c). For example, fluidics subsystem 110 may manage fluid for an irrigating cannula, infusion cannula, or other fluid delivery device. In some embodiments, fluidics subsystem 110 may be operatively coupled to a surgical cassette during a surgical procedure. For example, the surgical cassette may be inserted into, attached to, and/or integrated with fluidics subsystem 110 via a coupling mechanism. The coupling mechanism may comprise one or more of a latching mechanism, locking mechanism, or other similar connection mechanism. When fluidics subsystem 110 is operatively coupled to a surgical cassette, fluidics subsystem 110 may control irrigation and/or infusion of fluids through the surgical cassette, which may in turn be fluidically coupled to the one or more handpieces 112. In certain embodiments, the fluidics subsystem 110 includes one or more mechanical pumps having roller pump heads configured to engage with one or more corresponding pump assemblies on the surgical cassette, described below. The engagement of the roller pump heads and pump assemblies generates a source of pressure and/or vacuum utilized during an ophthalmic surgical procedure.

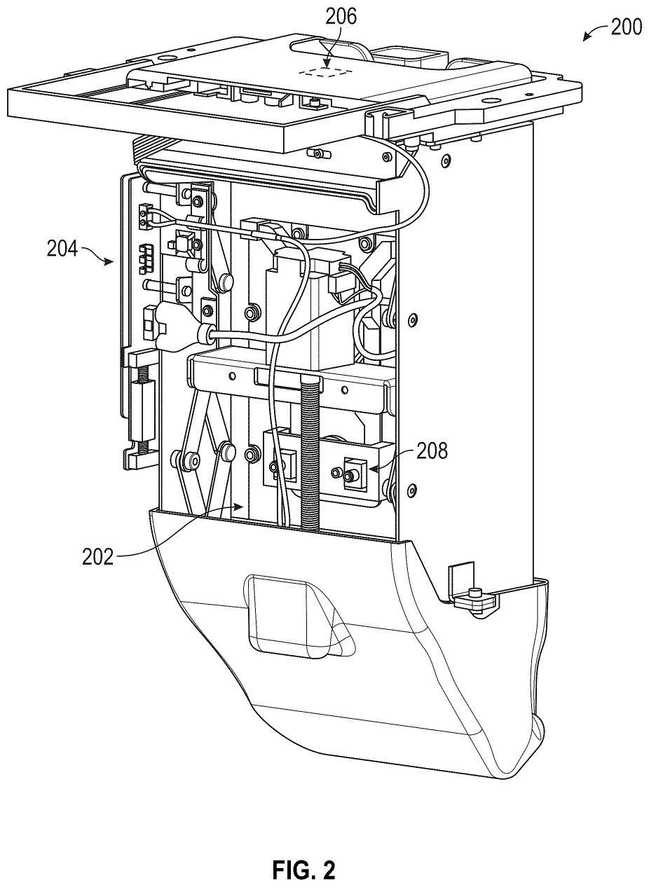

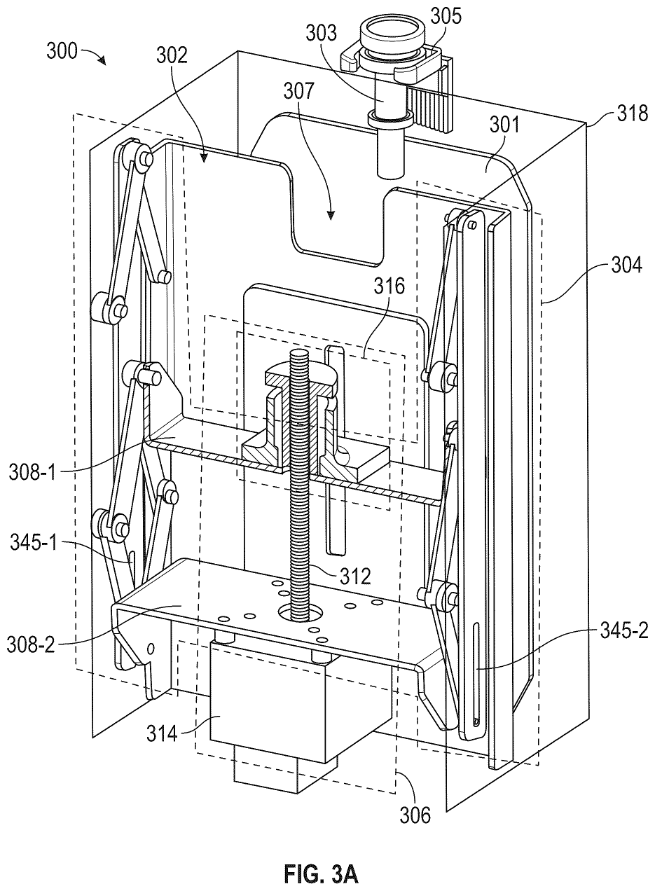

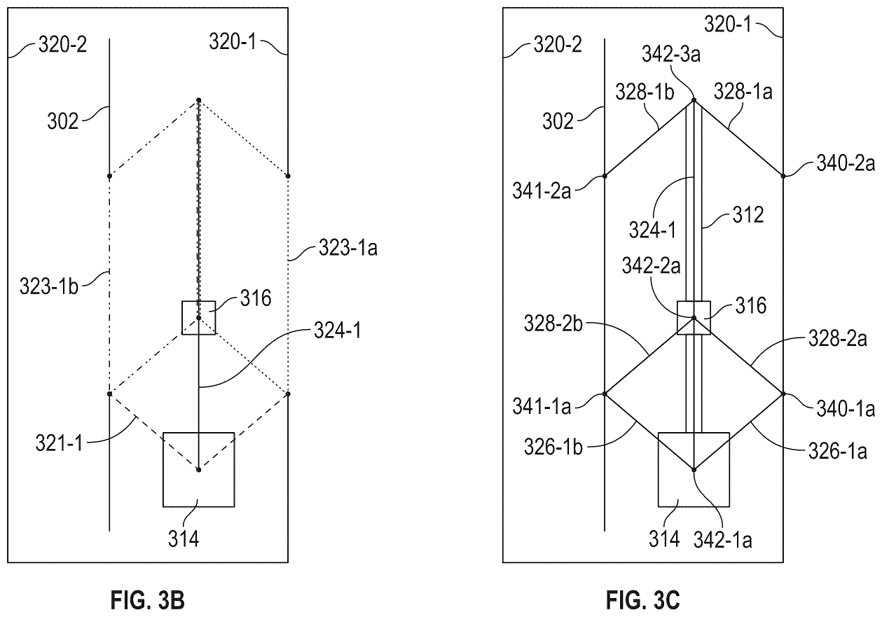

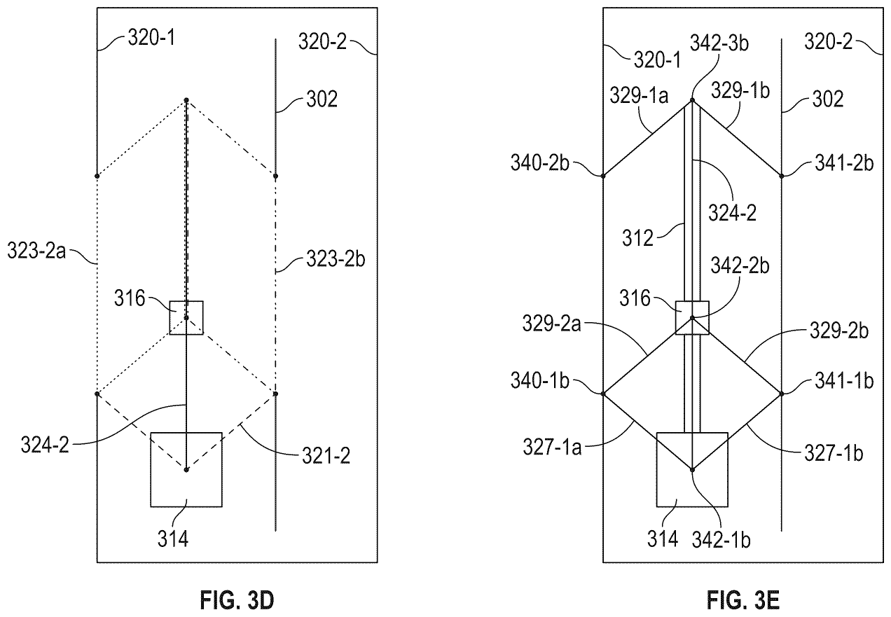

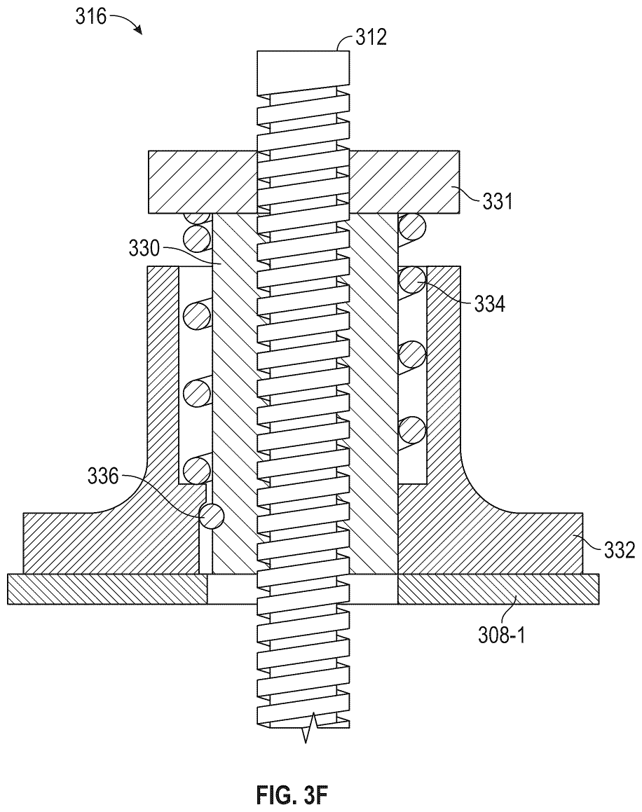

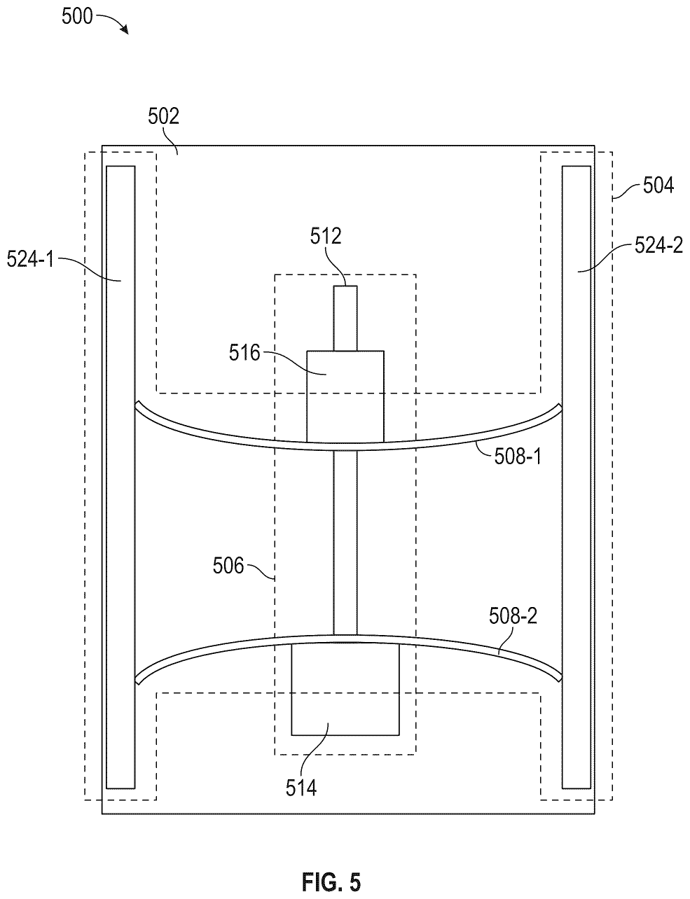

In certain embodiments, the fluidics subsystem 110 further comprises a fluidics module (shown as fluidics module 200 (FIG. 2)) with a (fluid bag) compression system (shown as compression system 202 (FIG. 2), 300 (FIG. 3A), and/or 500 (FIG. 5)) for applying pressure to a fluid bag loaded into the fluidics module 200 during an ophthalmic surgical procedure. The compression mechanism of the fluidics module 200 may control the irrigation and/or infusion of fluids routed through the surgical cassette. However, current compression mechanisms present a variety of limitations.

Current compression mechanisms typically require a large amount of space within the fluidics module 200, use a costly linear slide, and/or cannot provide backup pressure in the event of a power failure or other similar event. For example, in current systems, a drive mechanism including a lead screw and motor is typically oriented perpendicular to a squeeze plate that engages with a fluid bag. With this orientation, a large footprint within the fluidics module is needed to support lateral movement of the lead screw as driven by the motor. Additional space may also be needed to support a bag pressure sensor and its cabling, which is often disposed on a side of the fluid bag opposite the squeeze plate.

Accordingly, the systems described herein overcome many of the limitations associated with current fluidic systems.

Certain embodiments described herein provide improved fluidic systems for use during performance of ophthalmic procedures, including procedures involving irrigation and/or infusion of fluid inside a patient's eye. More particularly, certain embodiments provide fluidic systems that have a more compact design, are cost efficient, and facilitate backup irrigation pressure, which reduces complications associated with loss of power, motor fault, control fault, or other similar events affecting irrigation and/or infusion during ophthalmic procedures.

Certain embodiments of the present disclosure are directed to a compression system. In some embodiments, the system includes a squeeze plate configured to engage with a fluid bag, a linkage assembly, and a drive assembly. The linkage assembly comprises a plurality of brackets each coupled to a plurality of linkage sets. The drive assembly comprises a lead screw disposed through each of the plurality of brackets, an actuator configured to rotate the lead screw to actuate the drive assembly, and a nut assembly coupled to the lead screw and at least one of the plurality of brackets. In some embodiments, actuation of the linkage assembly causes lateral translation of the squeeze plate. Additionally, the nut assembly may be configured to provide a biasing force to maintain a target range of pressure on the fluid bag if the actuator fails.

FIG. 2 is a back side isometric view of a portion of an example fluidics module 200 of the console 100 of the ophthalmic surgical system 10 of FIG. 1A, according to certain embodiments. The fluidics module 200 may be implemented as part of the fluidics subsystem 110 in FIGS. 1A-1B.

In certain embodiments, the fluidics module 200 comprises a (fluid bag) compression system 202, a main printed circuit board assembly (PCBA) 204, a fluid bag identification (ID) camera 206, and a bag pressure sensor assembly 208. The main PCBA 204 may be mounted on a side of the compression system 202. The fluid bag ID camera 206 may be utilized to identify a fluid bag (shown as fluid bag 301 (FIG. 3A)) or the type of fluid bag, while the bag pressure sensor assembly 208 may be utilized to determine pressure within the fluid bag. In certain embodiments, the fluid bag ID camera 206 and/or the bag pressure sensor assembly 208 may be positioned on a mechanism side of the compression system 202 to help facilitate compact design of the compression system 202. The compression system 202 may be implemented as part of the console 100 for anterior and/or posterior eye surgery, in which the compression system provides irrigation and/or infusion pressure during ophthalmic procedure. The compression system 202 is described in further detail with reference to FIGS. 3A-3F.

FIG. 3A is a back side isometric view of an example compression system 300 of the fluidics module 200 of FIG. 2, according to certain embodiments. That is, the compression system 300 represents an embodiment of the compression system 202 of FIG. 2. The compression system 300 includes a squeeze plate 302, a linkage assembly 304, and a drive assembly 306 within a chamber 318. In certain embodiments, the compression system 300 may be configured to be disposed in surgical console 100. For example, the compression system 300 may be implemented in fluidics module 200 of surgical console 100 to control irrigation and/or infusion functions of the surgical console 100.

The compression system 300 may control irrigation and/or infusion by applying and/or maintaining pressure on a fluid bag 301 disposed within the fluidics module 200 during a surgical procedure. By applying pressure to the fluid bag 301, fluid may be directed out through a neck 303 of the fluid bag 301, and into a fluid line coupled to a surgical cassette or fluid delivery device. The neck 303 of the fluid bag 301 may be held by a fluid bag mount 305, which is disposed within the fluidics module 200.

The fluid bag 301 may contain a fluid or mixture of fluids used for ophthalmic procedures. For example, the fluid bag 301 may contain an infusion fluid and/or an irrigation fluid, which may be a balanced salt solution (BSS), a basic saline solution with a medication, a perfluorocarbon liquid, a viscoelastic substance, or other similar fluid.

The squeeze plate 302 is configured to engage and press against (e.g., “squeeze”) the fluid bag 301, thereby causing displacement of fluids from the fluid bag 301 (e.g., causing fluids to be flowed out of the fluid bag and to the patient). For example, the squeeze plate 302 can be laterally translated towards and/or away from the fluid bag 301 via actuation of the linkage assembly 304 and the drive assembly 306. The squeeze plate 302 may include a cutout 307 that corresponds to the neck 303 of the fluid bag 301. In certain embodiments, the cutout 307 prevents the squeeze plate 302 from applying pressure to the neck 303, which may dismount the fluid bag 301 and/or hinder irrigation and/or infusion.

The linkage assembly 304 includes a plurality of brackets: a first actuation bracket 308-1 and a second actuation bracket 308-2 (collectively referred to herein as “brackets 308” or “actuation brackets 308”). The brackets 308 may each be coupled to a plurality of linkage sets, which are best seen in FIGS. 3B and 3D. Although the linkage assembly 304 includes two brackets 308, the linkage assembly 304 may also include one bracket or more than two brackets.

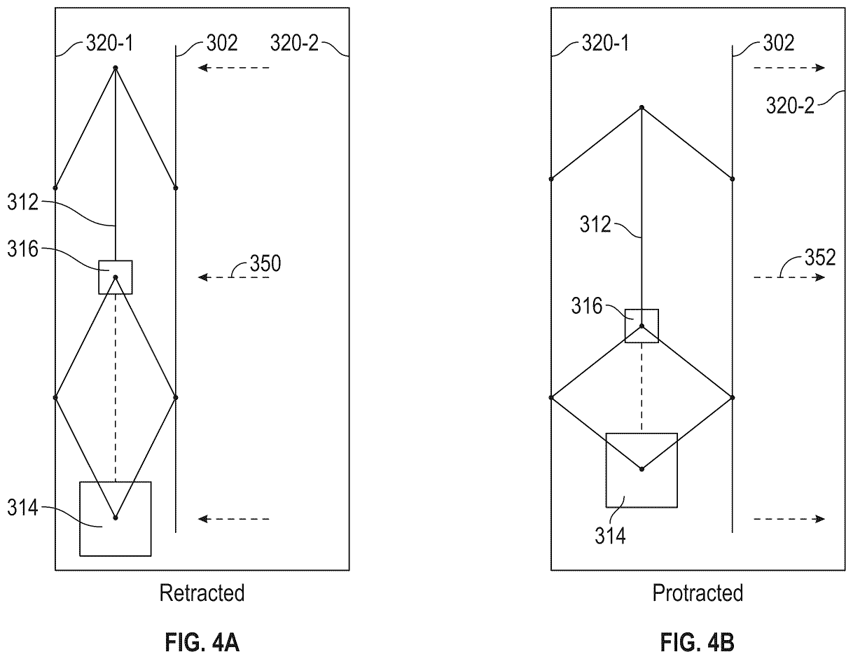

The drive assembly 306 includes a lead screw 312, an actuator 314, and a nut assembly 316. In certain embodiments, the lead screw 312 is disposed through each of the brackets 308 and may be rotated by the actuator 314. For example, the actuator 314 may be a motor configured to rotate the lead screw 312 in a first direction to protract the squeeze plate 302 toward the fluid bag 301, and/or in a second direction to retract the squeeze plate 302 away from the fluid bag 301. Other examples of the actuator 314 include a linear actuator, a rotary actuator, an electric motor (e.g., a servo motor or a stepper motor), or another similar actuator or motor configured to provide accurate, controlled rotation for precise movements. The drive assembly may also include more or less components for performing the same or similar function(s). The protraction and retraction of the squeeze plate 302 is described in further detail with reference to FIGS. 4A-4B.

In certain embodiments, the nut assembly 316, which is described in further detail with reference to FIG. 3F, is configured to provide a biasing force to maintain a target range of pressure on the fluid bag 301 if the actuator 314 fails. In other words, the nut assembly 316 incorporates a means for providing back up pressure to the fluid bag 301 if the actuator 314 fails. The actuator 314 may fail if, for example, there is a loss of power, a motor fault, a control fault, or other similar event that disables operation of the actuator 314.

In certain embodiments, the linkage assembly 304 and the drive assembly 306 are oriented parallel to the squeeze plate 302. By orienting the linkage assembly 304 and the drive assembly 306 parallel to the squeeze plate 302, the compression system 300 may be more compact and require less volume within the fluidics module 200 and the console 100.

With reference to FIGS. 2 and 3A, to help facilitate the compact design of the compression system 202 and/or 300, the fluid bag ID camera 206 and the bag pressure sensor assembly 208 may be positioned on a mechanism side of the compression system 202 and/or 300 (i.e., the drive assembly 306 side of the squeeze plate 302) to fit within the space around the linkage assembly 304 and the drive assembly 306. Additionally, the main PCBA 204 may be mounted on a side of the compression system 202 and/or 300 using a spring-loaded bracket assembly that facilitates connection of its blind mate connector to the console 100 when installed. Thus, the compactness of the compression system 202 and/or 300 allows for a smaller console (e.g., console 100 (FIG. 1A)), which creates more space in operating rooms where there is commonly a lack of available space due to large equipment.

The compression system 300 may have reduced associated fabrication costs as it eliminates the need for a precision linear slide. Further, fabrication costs may also be reduced by the compression system 300's compact design, which allows for smaller components and fewer materials.

FIGS. 3B-3E are schematic side views of the compression system 300 of FIG. 3A, according to certain embodiments.

Turning to FIG. 3B, FIG. 3B is a schematic left side view illustrating the plurality of linkage sets of the linkage assembly 304 of FIG. 3A. As shown in FIG. 3B, a left side of the linkage assembly 304 is comprised of a left side two-bar linkage set 321-1 and a plurality of left side four-bar linkage sets: a first left side four-bar linkage set 323-1a and a second left side four-bar linkage set 323-1b. The term left is used herein for clarity purposes to refer to the most left side of the compression system 300 when looking from the perspective shown in FIG. 3A but does not limit the orientation of the compression system 300.

Turning to FIG. 3C, the left side two-bar linkage set 321-1 includes a first bar 326-1a and a second bar 326-1b. The first bar 326-1a connects to a first inner surface 320-1 at a surface joint 340-1a, and the second bar 326-1b connects to the squeeze plate 302 at a squeeze plate joint 341-1a. The first inner surface 320-1 may be a wall that opposes the second inner surface 320-2, against which the fluid bag 301 may be disposed. Further, the first bar 326-1a and the second bar 326-1b share a common joint 342-1a.

At the common joint 342-1a, the left side two-bar linkage set 321-1 connects to the second actuation bracket 308-2 connected with the actuator 314. The common joint 342-1a may be disposed within a slot 345-1 (best seen in FIG. 3A) in the first vertical bracket 324-1, such that the left side two-bar linkage set 321-1 vertically slides (e.g., up and down) along a length of the slot 345-1 when the linkage assembly 304 is actuated. The slot 345-1 may provide stops for the left side two-bar linkage set 321-1 and help keep the linkage sets aligned.

The first left side four-bar linkage set 323-1a includes a first bar 328-1a and a second bar 328-2a, and the second left side four-bar linkage set 323-1b includes a first bar 328-1b and a second bar 328-2b. Both left side four-bar linkage sets 323-1a and 323-1b include the first vertical bracket 324-1, which acts as a common link and couples the linkage sets 323-1a and 323-1b. The first left side four-bar linkage set 323-1a connects to the first inner surface 320-1, while the second left side four-bar linkage set 323-1b connects to the squeeze plate 302.

The two left side four-bar linkage sets 323-1a and 323-1b share two common joints 342-2a and 342-3a. At one common joint 342-2a, the two left side four-bar linkage sets 323-1a and 323-1b connect to the first actuation bracket 308-1 connected with the nut assembly 316. The first bar 328-1a and the second bar 328-2a of the first left side four-bar linkage set 323-1a further connect to the first inner surface 320-1 at surface joints 340-1a and 340-2a, respectively. Whereas the first bar 328-1b and the second bar 328-2b of the second left side four-bar linkage set 323-1b further connect to the squeeze plate 302 at squeeze plate joints 341-1a and 341-2a, respectively.

Turning to FIG. 3D, FIG. 3D is a schematic right side view illustrating the plurality of linkage sets of the linkage assembly 304 of FIG. 3A. As shown in FIG. 3D, a right side of the linkage assembly 304 is comprised of a right side two-bar linkage set 321-2 and a plurality of right side four-bar linkage sets: a first right side four-bar linkage set 323-2a and a second right side four-bar linkage set 323-2b. The term right is used herein for clarity purposes to refer to the most right side of the compression system 300 when looking from the perspective shown in FIG. 3A, but does not limit the orientation of the compression system 300.

Turning to FIG. 3E, the right side two-bar linkage set 321-2 includes a first bar 327-1a and a second bar 327-1b. The first bar 327-1a connects to the first inner surface 320-1 at a surface joint 340-1b, and the second bar 327-1b connects to the squeeze plate 302 at a squeeze plate joint 341-1b. Further, the first bar 327-1a and the second bar 327-1b share a common joint 342-1b.

At the common joint 342-1b, the right side two-bar linkage set 321-2 connects to the second actuation bracket 308-2 connected with the actuator 314. The common joint 342-1b may be disposed within a slot 345-2 (best seen in FIG. 3A) in the second vertical bracket 324-2, such that the right side two-bar linkage set 321-2 vertically slides (e.g., up and down) along a length of the slot 345-2 when the linkage assembly 304 is actuated. The slot 345-2 may help keep the linkage sets vertically aligned. Further, the slot 345-2 may provide stops for the common joint 342-1b of the right side two-bar linkage set 321-2 to be pressed against, which helps prevent the squeeze plate 302 from being over protracted and/or retracted.

The first right side four-bar linkage set 323-2a includes a first bar 329-1a and a second bar 329-2a, and the second right side four-bar linkage set 323-2b includes a first bar 329-1b and a second bar 329-2b. Both right side four-bar linkage sets 323-2a and 323-2b include the first vertical bracket 324-1, which acts as a common link and couples the linkage sets 323-2a and 323-2b. The first right side four-bar linkage set 323-2a connects to the first inner surface 320-1, while the second right side four-bar linkage set 323-2b connects to the squeeze plate 302.

The two right side four-bar linkage sets 323-2a and 323-2b share two common joints 342-2b and 342-3b. At one common joint 342-2b, the two right side four-bar linkage sets 323-2a and 323-2b connect to the first actuation bracket 308-1 connected with the nut assembly 316. The first bar 329-1a and the second bar 329-2a of the first right side four-bar linkage set 323-2a further connect to the first inner surface 320-1 at surface joints 340-1b and 340-2b, respectively. Whereas the first bar 329-1b and the second bar 329-2b of the second right side four-bar linkage set 323-2b further connect to the squeeze plate 302 at squeeze plate joints 341-1b and 341-2b, respectively.

In certain embodiments, the joints 340, 341, and 342 of the linkage sets 321 and 323 may be a form of connection such as a pin, a bolt, laser or electron beam welding, or other similar connection mechanism. Additionally, the bars that connect to the first inner surface 320-1 may be stationary. The linkage sets 321 and/or 323 may also include less than or more than the bars associated with each linkage set 321 and/or 323.

FIG. 3F is a cross sectional view illustrating the lead screw 312 and the nut assembly 316 of the compression system 300 of FIG. 3A, according to certain embodiments. The nut assembly 316 comprises a nut 330 with a cap 331, a base unit 332, a spring 334 disposed between the nut 330 and the base unit 332, and at least one anti-rotation feature 336 disposed between the nut 330 and the base unit 332. As shown in FIG. 3F, the spring 334 may include a compression spring in series with the actuator 314, the nut 330, and the base unit 332. Although a compression spring is shown, other types of springs and other biasing devices are contemplated.

In certain embodiments, the nut 330 is configured to engage with the lead screw 312, and the base unit 332 couples the first actuation bracket 308-1 to the nut 330. The nut 330 also includes the cap 331 configured to engage with the base unit 332. Because the nut 330 and base unit 332 collectively couple the first actuation bracket 308-1 to the lead screw 312, rotation of the lead screw thereby actuates movement (e.g., vertical movement) of the first actuation bracket 308-1. Further, movement of the first actuation bracket 308-1 thereby actuates movement of the linkage sets 321 and 323, and the squeeze plate 302.

The anti-rotation feature 336 maintains rotational alignment between the nut 330 and the base unit 332 while allowing the base unit 332 and the first actuation bracket 308-1 to traverse longitudinally (e.g., extend vertically) when a biasing force, provided by the spring 334, is released. The anti-rotation feature 336 may be a bearing as shown in FIG. 3F, or may include a groove and a finger or other mechanism to maintain rotational alignment. Although the nut assembly 316 is shown as including one anti-rotation feature 336, the nut assembly 316 may include more than one anti-rotation feature 336. In certain embodiments, the nut assembly 316 includes three anti-rotation features. Additionally, the nut assembly 316 is not limited to the arrangement shown in FIG. 3F.

In certain embodiments, the actuator 314 is configured to create the biasing force provided by the spring 334. That is, the actuator 314 may create the biasing force by compressing the spring 334 during operation of the compression system 300. Compression of the spring 334 occurs by rotating the screw in a first rotational direction, which causes the nut 330 and the cap 331 to move downwards in a first linear direction towards the actuator 314, thereby compressing the spring 334 to a point where the cap 331 engages with the base unit 332 (i.e., bottoms out).

When (or if) the actuator 314 stops (e.g., due to failure of the actuator 314), the biasing force is released and provided via decompression of the spring 334. That is, when the biasing force is released, the first actuation bracket 308-1 and the base unit 332 transition from a compressed position to a decompressed position (shown in FIG. 3F) while the nut 330 remains in place. Release of the biasing force (i.e., decompression of the spring 334) causes the spring 334 to engage the cap 331 and the base unit 332, thereby causing the first actuation bracket 308-1 to continue to move downwards in the first linear direction. As the first actuation bracket 308-1 continues to move in the first linear direction towards the second actuation bracket 308-2, the linkage sets 321 and 323 continue to extend laterally and protract the squeeze plate 302 towards the fluid bag 301. Protracting the squeeze plate 302 towards the fluid bag 301 thereby maintains the target range of pressure on the fluid bag 301.

In certain embodiments, the target range of pressure on the fluid bag 301 may be maintained as infusion occurs, such that the target range of pressure is a safe range for a specific volume of fluid in the fluid bag 301. The target range of pressure on the fluid bag 301 may serve as backup pressure which slowly decays as fluid is dispensed from the fluid bag 301 and until the spring 334 is at its final decompressed position. In certain embodiments, the biasing force may be released manually (e.g., via input by a surgeon) or automatically (e.g., upon detection that the actuator 314 has failed).

By maintaining the target range of pressure on the fluid bag 301, irrigation of fluid may continue, for a given amount of time, during a power outage or other actuator failure event without sudden changes in pressure or fluid flow. As such, a stable environment may be maintained or prolonged within a patient's eye, which helps preserve the eye's shape and prevent complications associated with changes in intraocular pressure. Additionally, the surgeon may continue to perform the ophthalmic procedure while efforts are made to safely pause the procedure and/or resolve any mechanical issues affecting the compression system 300.

FIGS. 4A-4B are schematic side views of the compression system 300 of FIG. 3A illustrating lateral translation of a squeeze plate, according to certain embodiments.

Turning to FIG. 4A, a schematic right side view of the compression system 300 in a retracted position is shown. In FIG. 4A, the actuator 314 has rotated the lead screw 312 in a first direction (e.g., clockwise or counter-clockwise) to move the brackets 308 away from each other and retract the squeeze plate 302, as shown by arrows 350, away from a second inner surface 320-2 and the fluid bag 301. In other words, to place the system 300 in the retracted position, the brackets 308 may be moved away from each other, causing the bars of the linkage sets 321 and 323 collapse and/or move towards each other. In turn, this movement causes the squeeze plate 302 to be retracted from the second inner surface 320-2 and the fluid bag 301.

Turning to FIG. 4B, a schematic right side view of the compression system 300 in a protracted position is shown. In FIG. 4B, the actuator 314 has rotated the lead screw 312 in a second direction (e.g., counter-clockwise or clockwise) to move the brackets 308 towards each other to protract the squeeze plate 302, as shown by arrows 352, towards the second inner surface 320-2 and the fluid bag 301. In other words, to place the system 300 in the protracted position, the brackets 308 may be moved towards each other, causing the bars of the linkage sets 321 and 323 to expand and/or move away from each other, which in turn causes the squeeze plate 302 to be protracted towards the second inner surface 320-2 and the fluid bag 301.

The compression system 300 may also include a home position sensor, a bag pressure sensor assembly, a door closed sensor, and a bag ID camera assembly to monitor the movement and positioning of the squeeze plate 302. The home position sensor can determine a position of the linkage sets 321 and 323 by tracking a number of encoder pulses from when the squeeze plate 302 is at its retracted position (or home position), such that the home position sensor can determine when the squeeze plate 302 is at its retracted position, protracted position, or somewhere therebetween. The bag pressure sensor assembly may be pressed against the fluid bag 301 to determine the pressure of fluids within the fluid bag 301. The door closed sensor is positioned such that can detect when the door above the fluidics module 200 is closed. The bag ID camera may determine a serial number of the fluid bag 301 and/or a type of fluid bag (e.g., what fluids are within the fluid bag 301).

FIG. 5 is a cross sectional view of another example compression system 500 of the fluidics module 200 of FIG. 2, according to certain embodiments. That is, the compression system 500 represents an embodiment of the compression system 202 of FIG. 2. The compression system 500 includes a squeeze plate 502, a linkage assembly 504 with a first vertical bracket 524-1 and a second vertical bracket 524-2, and a drive assembly 506 with a lead screw 512 and an actuator 514 as described with reference to the compression system 300 of FIGS. 3A-3E.

However, as opposed to the nut assembly 316 providing the biasing force in the compression system 300, the compression system 500 includes a first spring bracket 508-1 and a second spring bracket 508-2 (collectively referred to herein as “spring brackets 508”) which provide the biasing force. As an example, the spring brackets 508 are comprised of a stainless steel material or other similar material configured to provide a biasing force.

As noted above, the actuator 514 rotates the lead screw 512, which causes a nut assembly 516 to traverse longitudinally (e.g., vertically up and down). Vertical movement of the nut assembly thereby causes a first spring bracket 508-1 and a second spring bracket 508-2 to move towards each other or away from each other. When moving down, after reaching a threshold force against the fluid bag (e.g., creating a certain threshold pressure on the fluid bag), inward flexure or bending of the spring brackets 508 toward each other may occur. The inward flexure or bending may result from the nut assembly 516 providing downward forces on the first spring bracket 508-1 and the actuator 514 providing upwards forces on the second spring bracket 508-2. This flexure of the spring brackets 508 provides the biasing force.

As such, the spring brackets 508 are configured to provide the biasing force if, for example, the actuator 514 fails. Release of the biasing force actuates the linkage assembly 504 and causes the squeeze plate 502 to maintain a target range of pressure on a fluid bag (e.g., fluid bag 301). When the biasing force is released, the spring brackets 508 transition from a flexed position (shown in FIG. 5) to a deflexed position while the actuator 514 and the nut assembly 516 remain in place. In other words, the release of the biasing force causes ends of the spring brackets 508 to move towards each other, which causes (e.g., actuates) a plurality of linkage sets to extend laterally and protract the squeeze plate 502 towards the fluid bag. Protracting the squeeze plate 502 towards the fluid bag thereby maintains the target range of pressure on the fluid bag.

Because the spring brackets 508 are configured to provide the biasing force, the nut assembly 516 of the compression system 500 may or may not also provide another biasing force. For example, the nut assembly 516 may comprise a nut, a base unit, a spring, and an anti-rotation feature as described with reference to the nut assembly 316 (FIG. 3F) or may comprise a nut and an optional base unit.

Further, the compression system 500 is not limited to the arrangement shown in FIG. 5 and may have fewer or more components in a different arrangement to perform the same or similar function(s).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. A compression system for a surgical console, the compression system comprising:
a squeeze plate configured to engage with a fluid bag;
a linkage assembly comprising a plurality of brackets each coupled to a plurality of linkage sets,
wherein actuation of the linkage assembly causes lateral translation of the squeeze plate; and
a drive assembly comprising:
a lead screw disposed through each of the plurality of brackets;
an actuator configured to rotate the lead screw to actuate the drive assembly; and
a nut assembly coupled to the lead screw and at least one of the plurality of brackets,
wherein the nut assembly is configured to provide a biasing force to maintain a target range of pressure on the fluid bag if the actuator fails.

2. The compression system of claim 1, wherein the nut assembly comprises:
a nut configured to engage with the lead screw;
a base unit that couples one of the plurality of brackets to the nut;
a spring disposed between the nut and the base unit; and
one or more anti-rotation features disposed between the nut and the base unit.

3. The compression system of claim 2, wherein:
the spring provides the biasing force when released; and
the one or more anti-rotation features prevent rotation of the nut when the biasing force is released.

4. The compression system of claim 2, wherein:
the spring provides the biasing force when released;
the release of the biasing force actuates the linkage assembly; and
the actuation of the linkage assembly causes the squeeze plate to maintain the target range of pressure on the fluid bag.

5. The compression system of claim 1, wherein the actuator is configured to:
rotate the lead screw in a first direction to move the plurality of brackets towards each other to protract the squeeze plate toward the fluid bag; and
rotate the lead screw in a second direction to move the plurality of brackets away from each other to retract the squeeze plate away from the fluid bag.

6. The compression system of claim 1, wherein the linkage assembly comprises:
a first actuation bracket coupled to a plurality of four-bar linkage sets coupled to an inner surface of the compression system, a plurality of common links, and the squeeze plate; and
a second actuation bracket with a plurality of two-bar linkage sets coupled to the inner surface of the compression system and the squeeze plate.

7. The compression system of claim 6, wherein the plurality of four-bar linkage sets comprises:
a first plurality of four-bar linkage sets that connect to a first vertical bracket, the first actuation bracket, the squeeze plate, and the inner surface of the compression system; and
a second plurality of four-bar linkage sets that connect to a second vertical bracket, the first actuation bracket, the squeeze plate, and the inner surface of the compression system.

8. The compression system of claim 7, wherein:
a first plurality of four-bar linkage sets comprises:
a first four-bar linkage set with:
a first bar of the first four-bar linkage set connected to the first vertical bracket and the inner surface of the compression system, and
a second bar of the first four-bar linkage set connected to the first vertical bracket, the first actuation bracket, and the inner surface of the compression system; and
a second four-bar linkage set with:
a first bar of the second four-bar linkage set connected to the first vertical bracket and the squeeze plate, and
a second bar of the second four-bar linkage connected to the first vertical bracket, the first actuation bracket, and the squeeze plate; and
a second plurality of four-bar linkage sets comprises:
a first four-bar linkage set with:
a first bar of the first four-bar linkage set connected to the second vertical bracket and the inner surface of the compression system, and
a second bar of first four-bar linkage set connected to the second vertical bracket, the first actuation bracket, and the inner surface of the compression system; and
a second four-bar linkage set with:
a first bar of the second four-bar linkage set connected to the second vertical bracket and the squeeze plate, and
a second bar of the second four-bar linkage set connected to the second vertical bracket, the first actuation bracket, and the squeeze plate.

9. The compression system of claim 6, wherein the plurality of two-bar linkage sets comprises:
a first two-bar linkage set that connects to the second actuation bracket, the squeeze plate, and the inner surface of the compression system; and
a second two-bar linkage set that connects to the second actuation bracket, the squeeze plate, and the inner surface of the compression system.

10. The compression system of claim 9, wherein:
the first two-bar linkage set comprises:
a first bar of the first two-bar linkage set connected to the second actuation bracket and the inner surface of the compression system, and
a second bar of the first two-bar linkage set connected to the second actuation bracket and the squeeze plate; and
the second two-bar linkage set comprises:
a first bar of the second two-bar linkage set connected to the second actuation bracket and the inner surface of the compression system, and
a second bar of the second two-bar linkage connected to the second actuation bracket and the squeeze plate.

11. A compression system for a surgical console, the compression system comprising:
a squeeze plate configured to engage with a fluid bag;
a linkage assembly comprising a plurality of brackets each coupled to a plurality of linkage sets,
wherein actuation of the linkage assembly causes lateral translation of the squeeze plate; and
a drive assembly comprising:
a lead screw disposed through each of the plurality of brackets;
an actuator configured to rotate the lead screw to actuate the drive assembly; and
a nut assembly coupled to the lead screw and at least one of the plurality of brackets,
wherein the plurality of brackets is configured to provide a biasing force to maintain a target range of pressure on the fluid bag if the actuator fails.

12. The compression system of claim 11, wherein at least one of the plurality of brackets is a spring bracket that provides the biasing force when released.

13. The compression system of claim 12, wherein:
the release of the biasing force actuates the plurality of linkage sets; and
the actuation of the plurality of linkage sets causes the squeeze plate to maintain the target range of pressure on the fluid bag.

14. The compression system of claim 12, wherein the actuator is configured to:
rotate the lead screw in a first direction to move the plurality of brackets towards each other to protract the squeeze plate toward the fluid bag; and
rotate the lead screw in a second direction to move the plurality of brackets away from each other to retract the squeeze plate away from the fluid bag.

15. The compression system of claim 11, wherein the linkage assembly and the drive assembly are oriented parallel to the squeeze plate.
